(19) United States
(12) Patent Application Publication
(10) Pub. No.: US 2021/0371386 A1
(43) Pub. Date: Dec. 2, 2021

(54) MANUFACTURE OF HYDRAZINYL COMPOUNDS USEFUL IN THE MANUFACTURE OF PYRAZOLE CARBOXYLIC ACID AND DERIVATIVES, HYDRAZINYL COMPOUNDS AND THEIR USE

(71) Applicant: SOLVAY SA, Brussels (BE)

(72) Inventor: Janis JAUNZEMS, Hannover (DE)

(21) Appl. No.: 16/322,649

(22) PCT Filed: Jul. 28, 2017
(86) PCT No.: PCT/EP2017/069239

§ 371 Date: Feb. 1, 2019

(30) Foreign Application Priority Data
Aug. 2, 2016 (EP) 16182310.9
Apr. 5, 2017 (EP) 17165046.8

(51) Int. Cl. C07D 231/14; C07C 241/02; C07D 231/12

(52) U.S. Cl. CPC C07D 231/14; C07D 231/12; C07C 241/02

(57) ABSTRACT

The present invention concerns the manufacture of hydrazinyl compounds useful in the manufacture of pyrazole carboxylic acid and derivatives thereof and processes for the manufacture of agrochemical or pharmaceutical compounds. The invention also concerns hydrazinyl compounds and their use.

This application claims priority to EP applications No. 16182310.9 and 17165046.8, the whole content of this application being incorporated herein by reference for all purposes.

The present invention concerns the manufacture of hydrazinyl compounds useful in the manufacture of pyrazole carboxylic acid and derivatives thereof, hydrazinyl compounds, their use and processes for the manufacture of agrochemical and pharmaceutical compounds.

3-halomethylpyrazol-4-yl carboxylic acids and esters are valuable intermediates in the synthesis of agrochemical and pharmaceutical active ingredients. Agrochemical active ingredients which contain such pyrazol building blocks are, for example, 2′-[1,1′-bicycloprop-2-yl]-3-(difluoromethyl)-1-methylpyrazole-4-carboxanilide (Sedaxane), as described, for example, in WO2006015866, 3-(difluoromethyl)-1-methyl-N-[2-(3′,4′,5′-trifluorophenyl)phenyl]pyrazole-4-carboxamide (Fluxapyroxad), as described, for example, in WO2006087343, N-(3′,4′-Dichloro-5-fluorobiphenyl-2-yl)-3-(difluoromethyl)-1-methylpyrazole-4-carboxamide (Bixafen), as described, for example, in WO2003070705, 3-(Difluoromethyl)-1-methyl-N-[1,2,3,4-tetrahydro-9-(1-methylethyl)-1,4-methanonaphthalen-5-yl]-1H-pyrazole-4-carboxamide (Isopyrazam), as described, for example, in WO2004035589, (RS)—N-[9-(Dichloromethylen)-1,2,3,4-tetrahydro-1,4-methanonaphthalin-5-yl]-3-(difluoromethyl)-1-methyl-1H-pyrazole-4-carboxamide (Benzovindiflupyr), as described, for example, in WO07048556. Generally, 3-halomethylpyrazol-4-yl carboxylic acids, often obtained by hydrolysis of their esters, are converted into the carboxamides, for example after conversion into the 3-halomethylpyrazol-4-yl carboxylic acid halide. Other conversions, wherein the carboxamide is generated directly from the ester or acid, have also been described, such as in WO2012055864 and WO 2007/031323. All foregoing cited patent applications are hereby incorporated for all purposes.

EP2247577 B1 describes the regioselective manufacture of 3-halomethylpyrazol-4-yl carboxylic esters starting from 2-(aminomethylidene)-3-oxobutyric esters.

Object of the present invention is to provide hydrazinyl compounds useful in the manufacture of pyrazole carboxylic acid and derivatives, to provide processes for the manufacture of hydrazinyl compounds useful in the manufacture of pyrazole carboxylic acid and derivatives, use of hydrazinyl compounds for the manufacture of an agrochemical or pharmaceutical compound or their intermediates, and a process for the manufacture of an agrochemical or pharmaceutical compound or their intermediates. These and other objects are achieved by the invention as outlined in the description and claims.

The present invention concerns a process for the manufacture of a compound according to formula (I),

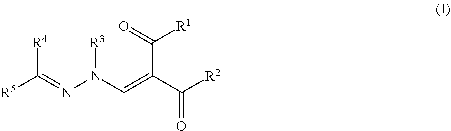

The invention further concerns a process for the manufacture of a compound according to formula (II).

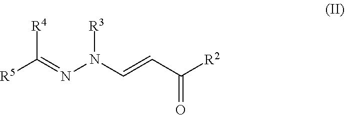

The invention further concerns a process for the manufacture of a compound according to formula (VI)

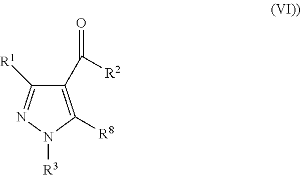

which comprises a step of performing at least one of the processes for the manufacture of a compound of formula (I) and (II).

Further embodiments of the present invention are the compounds of formula (II), the compounds of formula (I), use of at least one of compounds of formula (I) and formula (II) for the manufacture of an agrochemical or pharmaceutical compound or their intermediates, and a process for the manufacture of an agrochemical or pharmaceutical compound or their intermediates, which comprises a step of performing at least one of the processes of manufacturing compound (I) and compound (II).

In the present invention, designations in singular are in intended to include the plural; for example, “a solvent” is intended to denote also “more than one solvent” or “a plurality of solvents”.

In the context of the present invention, the term “comprising” is intended to include the meaning of “consisting of”.

When a double bond is depicted in a particular E/Z geometry, this is intended to also denote the other geometric form as well as mixtures thereof.

In a first embodiment of the present invention, the invention concerns a process for the manufacture of a compound according to formula (I),

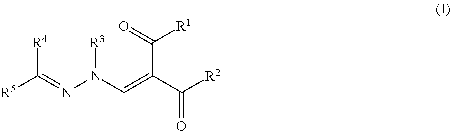

which comprises a step of reacting a compound of formula (II)

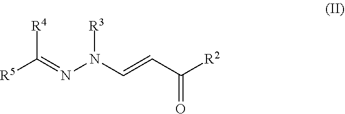

with a compound of formula (III)

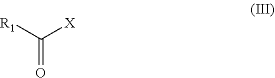

wherein

R¹ is selected from C₁-C₄-alkyl groups which may be substituted by one, two or three halogen atoms selected from the group consisting of F, Cl and Br or by a CF₃ group;

R² is selected from the group consisting of C₁-C₈-alkyl, aryl, C₃-C₈ cycloalkyl, aralkyl and heteroaryl, each of which is optionally substituted;

R³ is selected from the group consisting of C₁-C₁₂-alkyl, C₂-C₆ alkenyl, C₃-C₈ cycloalkyl, aryl, heteroaryl, aralkyl, each of which is optionally substituted; or R³ is a nitrogen protecting group;

R⁴ and R⁵ independently from each other are selected from the group consisting of H; C₁-C₁₂-alkyl, C₃-C₈ cycloalkyl which optionally contains one or two heteroatoms selected from the group consisting of N, O and S, aryl and heteroaryl, each of which is optionally substituted; or R⁴ and R⁵ together with the carbon atom to which they are attached form a 4, 5 or 6-membered cycloalkyl, which optionally contains one or two heteroatoms selected from the group consisting of N, O and S, aryl or heteroaryl group, each of which is optionally substituted;

X is a halogen atom, preferably X is F, Br or Cl; or with a respective anhydride (Ma) of the compound of formula (III).

For the purpose of the present invention, the definition C₁-C₁₂-alkyl, or sub-ranges thereof, such as a C₁-C₄ or C₁-C₈ alkyl group, comprises the largest range defined herein for an alkyl group. Specifically, this definition comprises, for example, the meanings methyl, ethyl, n-propyl, isopropyl, n-, iso-, sec- and t-butyl, n-pentyl, n-hexyl, 1,3-dimethylbutyl, 3,3-dimethylbutyl, n-heptyl, n-nonyl, n-decyl, n-undecyl and n-dodecyl. Often, methyl, ethyl, n-propyl, isopropyl, n-, iso-, sec- and t-butyl are most preferred residues selected from the group C₁-C₁₂-alkyl.

R¹ is selected from C₁-C₄-alkyl groups which may be substituted by one, two or three halogen atoms selected from the group consisting of F, Cl and Br or by a CF₃ group. Preferably, R¹ is methyl which is substituted by at least one fluorine atom. In another preferred embodiment, R¹ may also be ethyl which is substituted by at least one fluorine atom. More preferably, R¹ is selected from the group consisting of CF₂Cl, CF₂H, CFCl₂, CFClH, CF₂Br, CF₂CF₃ and CF₃. CF₂H and CF₃ are the most preferred groups R¹.

R² is selected from the group consisting of C₁-C₈-alkyl group, aryl, C₃-C₈ cycloalkyl, aralkyl and heteroaryl, each of which is optionally substituted.

The term “C₃-C₁₀-cycloalkyl” or “C₃-C₈-cycloalkyl”, as used in this invention, denotes mono-, bi- or tricyclic hydrocarbon groups comprising 3 to 10 or 3 to 8 carbon atoms, especially 3 to 6 carbon atoms. Examples of monocyclic groups include cyclopropyl, cyclobutyl, cyclopentyl, cyclohexyl, cycloheptyl or cyclooctyl. Examples of bicyclic groups include bicyclo[2.2.1]heptyl, bicyclo[3.1.1]heptyl, bicyclo[2.2.2]octyl and bicyclo[3.2.1]octyl. Examples of tricyclic groups are adamantyl and homoadamantyl. A cycloalkyl group can optionally contain one or two heteroatoms selected from the group consisting of N, O and S.

In the context of the present invention, aryl denotes, unless defined otherwise, aromatic hydrocarbon groups which may contain one, two or more heteroatoms selected from the group consisting of O, N, P and S and which may optionally be substituted by further groups selected from the group consisting of R′, —X′, —OR′, —SR′, —NR′₂, —SiR′₃, —COOR′, —(C—O)R′, —CN and —CONR′₂, where R′ and X′ are defined as above.

In one aspect, the term “aryl” is a C₅-C₁₈-aryl. The term “C₅-C₁₈-aryl” denotes the largest range defined herein for an aryl groups having 5 to 18 skeleton atoms, where the carbon atoms may be replaced by heteroatoms, thus forming a “heteroaryl”. Specifically, this definition comprises, for example, the meanings cyclopentadienyl, phenyl, cycloheptatrienyl, cyclooctatetraenyl, naphthyl and anthracenyl; 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl; 1-pyrrolyl, 1-pyrazolyl, 1,2,4-triazol-1-yl, 1-imidazolyl, 1,2,3-triazol-1-yl, 1,3,4-triazol-1-yl; 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

In the context of the present invention, arylalkyl (abbreviated “aralkyl”) denotes, unless defined otherwise, alkyl groups which are substituted by aryl groups, which may have a C₁₋₈-alkylene chain and which may be substituted in the aryl skeleton or the alkylene chain by one or more heteroatoms selected from the group consisting of O, N, P and S and optionally by further groups selected from the group consisting of R′, —X′, —OR′, —SR′, —NR′₂, —SiR′₃, —COOR′, —(C—O)R′, —CN and —CONR′₂, where R′, which may further contain one or more heteroatoms selected from the group consisting of N, O, P and S, and X′ are defined as above.

The definition for C₇-C₁₉-aralkyl comprises the largest range defined herein for an arylalkyl group having a total of 7 to 19 atoms in the skeleton and the alkylene chain. Specifically, this definition comprises, for example, the meanings benzyl and phenylethyl.

In the context of the present invention, alkylaryl (abbreviated “alkaryl”) denotes unless defined otherwise, aryl groups which are substituted by one or more alkyl groups, or which may have a C₁-C₈-alkane diyl chain, and which may be substituted in the aryl skeleton, the one or more alkyl groups or the alkane diyl chain by one or more heteroatoms selected from the groups consisting of O, N, P and S and optionally by further groups selected from the group consisting of R′, —X′, —OR′, —SR′, —NR′₂, —SiR′₃, —COOR′, —(C—O)R′, —CN and —CONR′₂, where R′, which may further contain one or more heteroatoms selected from the group consisting of N, O, P and S, and X′ are defined as above.

The definition for C₇-C₁₉-alkylaryl comprises the largest range defined herein for an alkylaryl group having a total of 7 to 19 atoms in the skeleton and the alkyl chain or alkane diyl chain. Specifically, this definition comprises, for example, the meanings tolyl, 2,3-, 2,4-, 2,5-, 2,6-, 3,4- or 3,5-dimethylphenyl.

The term “C₂-C₆-alkenyl group “denotes a group comprising a carbon chain and at least one double bond. Alkenyl group are, for example, ethenyl, propenyl, butenyl, pentenyl or hexenyl.

According to the present invention, alkyl, aralkyl, aryl, heteroaryl or cycloalkyl groups are optionally substituted. In this embodiment, said groups may be substituted by one or more substituents selected from the group consisting of —R′, —X′, —OR′, —SR′, —NR′₂, —SiR′₃, —COOR′, —(C—O)R′, —CN and —CONR′₂, in which R′ are selected independently, wherein R′ is hydrogen or a C₁-C₁₂-alkyl group and X′ is F, Cl, Br, or I.

According to the present invention, R³ is selected from the group consisting of C₁-C₁₂-alkyl, C₂-C₆ alkenyl, cycloalkyl, aryl, heteroaryl, aralkyl, each of which is optionally substituted; or R³ is a nitrogen protecting group. Most preferably, R³ is a methyl group. In another aspect, R³ is a cycloalkyl or aryl group, all of which may optionally be substituted. The cycloalkyl or aryl group may be attached to the nitrogen via an alkyl diyl bridge, such as a —CH₂— or —CH₂—CH₂— group.

The term “nitrogen protecting group” intends to denote a group that is not cleaved by each of the reactions in the manufacturing method of the present invention, and is cleaved by other chemical methods (e.g., chemical methods such as hydrogenolysis, hydrolysis, electrolysis, photolysis as generally used in organic synthetic chemistry) into the N—H. Such protecting group can be selected from the commonly known or even well-known protecting groups known as amino-protecting groups. Examples include: alkyl carbamate based protecting groups such as tert-butyldiphenylsilyl, t-butyldimethylsilyl, methoxycarbonyl, ethoxycarbonyl, tert-butoxycarbonyl (Boc) groups; arylalkyl carbamate based protecting groups such as 9-fluorenylmethyloxycarbonyl (Fmoc); aryl sulfonamide based protecting groups such as benzenesulfonyl, p-toluenesulfonyl (Ts) group; amide based protecting groups such as carboxamido, acetamido, trifluoroacetamide (TFA), commonly known to persons skilled in the art according to synthetic chemistry reference books such as the “Protective Groups in Organic Synthesis” (T.W. Greene et. al, John Wiley & Sons, inc).

According to the present invention, R⁴ and R⁵ independently from each other are selected from the group consisting of H; C₁-C₁₂-alkyl group, C₃-C₈ cycloalkyl which optionally contains one or two heteroatoms selected from the group consisting of N, O and S, aryl and heteroaryl, each of which is optionally substituted; or R⁴ and R⁵ are together with the carbon atom to which they are attached form a 4, 5 or 6-membered cycloalkyl, which optionally contains one or two heteroatoms selected from the group consisting of N, O and S, aryl or heteroaryl group, each of which is optionally substituted. In one preferred aspect, R⁴ is a hydrogen atom and R⁵ is selected from the group consisting of C₁-C₁₂-alkyl, C₃-C₈ cycloalkyl, aryl and heteroaryl, each of which is optionally substituted. More preferably, R⁴ is H and R⁵ is an aryl group, more preferably a phenyl group. When R⁴ is a phenyl group, and when the phenyl group is substituted, the phenyl group is most preferably substituted by an NR′₂ or OR′ group.

According to the present invention, X is a halogen atom, preferably X is F, Br or Cl. In one preferred aspect, X in (III) is F or Cl, preferably, F, and R¹ is CF₂H or CF₃.

In the process of manufacturing a compound of formula (I), compound (III) can also be replaced by a corresponding anhydride of formula (Ma), such as (R¹—O—)₂CO. For example, CF₃C(O)F can be replaced by CF₃C(O)—O—(O)CF₃, and CF₂HC(O)F can be replaced by HF₂C—C(O)—O—(O)C—CF₂H. It is preferred to apply a compound of formula (III) which embodiment is explained now in further detail.

In one aspect, the reaction between (II) and (III) is carried out in the presence of a base. Bases suitable for this purpose are organic bases, for example the abovementioned acyclic tertiary amines, such as trimethylamine, triethylamine, diisopropylethylamine, tert-butyldimethylamine or ethyldicyclohexylamine, the abovementioned cyclic tertiary amines, such as N-methylpyrrolidine, N-methylpiperidine, N-methylmorpholine, N,N′-dimethylpiperazine, pyridine, collidine, lutidine or 4-dimethylaminopyridine, or bicyclic amines, such as diazabicycloundecene (DBU) or diazabicyclononene (DBN). Trimethylamine, triethylamine, diisopropylethylamine are preferred, and trimethylamine is particularly preferred. Also suitable as bases are inorganic compounds, for example alkali metal and alkaline earth metal hydroxides, such as sodium hydroxide, potassium hydroxide or calcium hydroxide, alkali metal and alkaline earth metal oxides, such as lithium oxide, sodium oxide, calcium oxide or magnesium oxide, alkali metal and alkaline earth metal carbonates, such as lithium carbonate or calcium carbonate, alkali metal bicarbonates, such as sodium bicarbonate, alkali metal and alkaline earth metal hydrides, such as lithium hydride, sodium hydride, potassium hydride or calcium hydride, or alkali metal amides, such as lithium amide, sodium amide or potassium amide. Suitable bases are also alcoholates.

The invention further concerns a process for the manufacture of a compound according to formula (II),

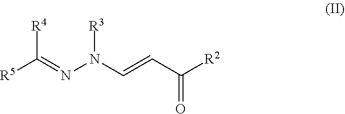

which comprises the step of reacting a compound of formula (IV) and formula (V)

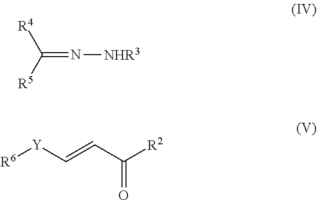

wherein

R¹, R², R³, R⁴ and R⁵ have the same meaning as above, Y is selected from the group consisting of S, O and NR⁷, wherein 0 and NR⁷ are preferred,

R⁷ and R⁶ independently are selected from the group consisting of C₁-C₁₂-alkyl, C₂-C₆ alkenyl or C₃-C₁₀-cycloalkyl, each of which is optionally substituted, or, when Y═NR⁶, R⁶ together with R⁷ and the nitrogen atom to which the two radicals are attached are an optionally substituted 5- to 10-membered heterocyclic radical which, in addition to the nitrogen atom, may contain further 1, 2 or 3 heteroatoms selected from the group consisting of O, N and S as ring members. In one particular aspect, Y is S, wherein R⁶ is hydrogen or a C₁-C₁₂-alkyl group, preferably a C₁-C₄-alkyl group. In another particular aspect, Y is 0, wherein R⁶ is hydrogen or a C₁-C₁₂-alkyl group, preferably a C₁-C₄-alkyl group. Generally, and in particular in connection with the definition of the group —NR⁶R⁷, the term “5- to 10-membered heterocyclic radical” denotes a nitrogenous mono- or bicyclic group having 5, 6, 7, 8, 9 or 10 ring members, which is attached via the nitrogen atom to the remainder of the compound of the formula (I) or (II), which, in addition to the nitrogen atom, may have a further 1, 2 or 3 heteroatoms selected from the group consisting of O, N and S as ring members and which is unsubstituted or may have 1, 2 or 3 substituents. The substituents, provided they are attached to a carbon atom of the heterocyclic radical, are preferably selected from the group consisting of halogen, CN, C₁-C₄ alkyl, C₁-C₄-haloalkyl, C₁-C₄-alkoxy and C₁-C₄-haloalkoxy and, provided they are attached to a further nitrogen atom of the heterocyclic radical, are preferably selected from the group consisting of C₁-C₄-alkyl and C₁-C₄-haloalkyl. Examples of 5- to 10-membered heterocyclic radicals are pyrrol-1-yl, pyrrolidin-1-yl, oxazolidin-3-yl, thiazolidin-3-yl, imidazol-1-yl, imidazolin-1-yl, 3-methylimidazolin-1-yl, 3-ethylimidazolin-1-yl, 3-propylimidazolin-1-yl, 3-(1-methylethyl)imidazolin-1-yl, 3-butylimidazolin-1-yl, 3-(1,1-dimethylethyl)imidazolin-1-yl, pyrazol-1-yl, pyrazolidin-1-yl, 2-methylpyrazolidin-1-yl, 2-ethylpyrazolidin-1-yl, 2-propylpyrazolidin-1-yl, 2-(1-methylethyl)pyrazolidin-1-yl, 2-butylpyrazolidin-1-yl, 2-(1,1-dimethylethyl)pyrazolidin-1-yl, piperidin-1-yl, morpholin-4-yl, thiamorpholin-4-yl, piperazin-1-yl, 4-methylpiperazin-1-yl, 4-ethylpiperazin-1-yl, 4-propylpiperazin-1-yl, 4-(1-methylethyl)piperazin-1-yl, 4-butylpiperazin-1-yl, 4-(1,1-dimethylethyl)piperazin-1-yl, indol-1-yl, indolin-1-yl, isoindol-1-yl, isoindolin-1-yl, indazol-1-yl, indazolin-1-yl, 2-methylindazolin-1-yl, indazolin-2-yl and 1-methylindazolin-1-yl; the heterocyclic groups mentioned above are unsubstituted, or 1, 2 or 3 of the ring carbon atoms carry a substituent selected from the group consisting of halogen, CN, nitro, C₁-C₄-alkyl, C₁-C₄-alkoxy and C₁-C₄-haloalkoxy. Preferred heterocyclic radicals are optionally substituted piperidinyl and optionally substituted morpholinyl. This process of manufacture of compound (II) has the advantage, especially when combined with the process for the manufacture of compound (I), that it allows to efficiently recover compounds of R⁶YH, which are commonly released in the process for the manufacture of (II). This is especially advantageous if Y is NR⁷ and additional nitrogenous bases such as triethylamine are used in further process steps, which will give a difficult to separate and/or recoverable mixture in later process steps. Preferably, when Y is 0, then R⁶ is methyl or ethyl. Preferably when Y is NR⁷, R⁷ is methyl or ethyl.

The compounds of formula (III) are known to be carboxylic acid halides. Many compounds falling under the formula (III) are well established and commercially available. The manufacture of difluoroacetyl fluoride is, for example, disclosed in EP694523 and U.S. Pat. No. 5,905,169 which are hereby incorporated by reference for all purposes. The manufacture of difluorochloroacetyl chloride is, for example, disclosed in U.S. Pat. No. 5,545,298 or 5,569,782, which are hereby incorporated by reference for all purposes, as well as the manufacture of trifluoroacetyl chloride. Compounds of formula (V) are known in the literature, for example when Y is 0 and R⁶ is Et, R² being CF₃, (V) is 4-ethoxy-1,1,1-trifluoro-3-buten-2-one (ETFBO), which manufacture is known to the person skilled in the art, e.g. from WO2010000871 (ETFBO), which is hereby incorporated by reference for all purposes, or Tietze, L. F. et al, Organic Syntheses, 69, 238-244; 1990 (4-ethoxy-1,1,1-trichloro-3-buten-2-one ETCBO, with R² being CCl₃). When Y in (Y) is NR⁷, (V) can be obtained, for example, from (V) when Y is 0 by addition of the respective compound NHR⁶R⁷.

In one aspect, (II) is obtained by reacting a compound of formula (IX) and a compound of formula (IV), in particular when R³, R⁴ in (IV) is H and R⁵ is optionally substituted phenyl. M in the formula (IX) is a metal ion, for example an alkali or earth alkali cation. Depending on the valency of the metal ion, one or more organic residues of the formula (IX) are present. M often is an alkali metal cation, preferably sodium.

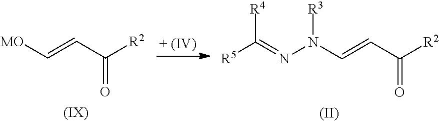

The compound of formula (IX) can be obtained, for example, by reaction of methyl formate and acetone in the presence of sodium methoxide. (IX) can also be obtained, for example, by reaction of acetic ethyl ester (when R^{e} is CH₃) with acetone in the presence of sodium methoxide. This method to obtain (II) can be combined with any of the processes of the present invention.

Manufacturing of (V) when Y is NR⁷ is also known from CN101260062, JP2006298873 or US20100317655, all of which are hereby incorporated by reference for all purposes. Compounds of formula (IV) have been described, for example, in the literature (Zhurnal Organicheskoi Khimii (1968), 4(6), 986-92) and can be obtained by reacting commercially available hydrazines H₂N—NHR₃ with carbonyl compounds R⁴R⁵C(O).

The invention further concerns a process for the manufacture of a compound of formula (VI)

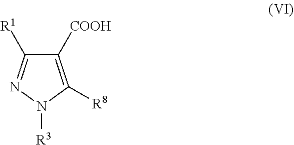

wherein R¹ and R³ have the same meaning as above, and R⁸ is selected from the group consisting of H, X′, COOR′, OR′, SR′, C(O)NR′₂, wherein is hydrogen or a C₁-C₁₂-alkyl group, CN, C₁-C₁₂-alkyl, C₂-C₆ alkenyl, aryl, cycloalkyl, aralkyl, heteroaryl, each of which is optionally substituted, with the proviso that both R′ in C(O)NR′₂ may be the same or different, which comprises a step of performing at least one of the processes for the manufacture of compounds of formula (I) or (II) as described above.

In one particular embodiment, the process for the manufacture of (VI) comprises additionally at least one of the steps of

(a) adding an acidic compound, preferably selected from the group consisting of HCl, H₂SO₄, KHSO₄ and HF, to the compound of formula (I) to obtain a compound of formula (VII)

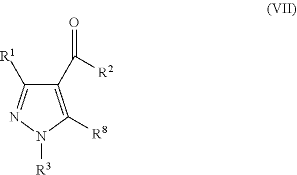

(b) reacting a compound of formula (VII) with at least one oxidizing agent, preferably with at least one oxidizing agent in the presence of at least one base

wherein R¹, R³, R⁸ and R² have the same meaning as above.

In step (b), the at least one oxidizing agent is preferably selected from the group consisting of halogen, oxyacids of halogen and salts thereof, a peroxide, molecular oxygen molecular and ozon, wherein halogen and aqueous solutions of salts hypohalogenous acids are more preferred, and sodium hypochlorite and sodium hypobromite are particularly preferred.

When at least one base is present in step b), the at least one base preferably is selected from the group consisting of earth alkaline or alkaline metal bases, such as hydroxides, carbonates, bicarbonates, oxides, amides or hydrides, for example sodium hydroxide, potassium hydroxide or calcium hydroxide, lithium oxide, sodium oxide, calcium oxide, magnesium oxide, lithium carbonate, calcium carbonate, sodium bicarbonate, lithium hydride, sodium hydride, potassium hydride, calcium hydride, lithium amide, sodium amide or potassium amide. When at one base is present in step b), generally a carboxylate compound (VIII) is formed, with one counter anion M⁺ or M²⁺, in which case two carboxylates are present per M²⁺, wherein M is an earth alkaline or alkaline metal cation.

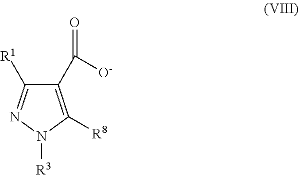

The carboxylate compound (VIII) can be transformed into the compound of formula (VI) by reaction with a suitable acid, such as a mineral acid, preferably HCl.

The invention further concerns compounds of formula (II), wherein R², R³, R⁴ and R⁵ have the same meaning as given above, and compounds of formula (I), wherein R¹, R², R³, R⁴ and R⁵ have the same meaning as given above. Preferred embodiments of R¹, R², R³, R⁴ and R⁵ are also preferred embodiments for the compounds of formulae (II) and (I).

Another aspect of the present invention is the use of at least one of compounds of formula (I) and formula (II) for the manufacture of an agrochemical or pharmaceutical compound or their intermediates.

The invention also concerns a process for the manufacture of an agrochemical or pharmaceutical compound or their intermediates, which comprises at least one of the processes given above and according to any one of claims 1 to 10. The process often comprises a further step of converting (VII) into its corresponding acid chloride, and reaction of the formed acid chloride with an aniline to form an agrochemically active carboxamide compound. Such a reaction is known, for example, from WO2003070705. In such a process for the manufacture of an agrochemical compound, for example compounds such as N-(3′,4′-Dichlor-5-fluorbiphenyl-2-yl)-3-(difluormethyl)-1-methylpyrazol-4-carboxamid, 3-(difluoromethyl)-1-methyl-N-[2-(3′,4′,5′-trifluorophenyl)phenyl]pyrazole-4-carboxamide, N-(2-Bicyclopropyl-2-ylphenyl)-3-difluoromethyl-1-methyl-1H-pyrazol-4-carboxylic acid amide, 3-(Difluormethyl)-1-methyl-N-[1,2,3,4-tetrahydro-9-(1-methylethyl)-1,4-methanonaphthalen-5-yl]-1H-pyrazol-4-carboxamid or N-[(1RS,4SR)-9-(dichloromethylidene)-1,2,3,4-tetrahydro-1,4-methanonaphthalen-5-yl]-3-(difluoromethyl)-1-methyl-1H-pyrazole-4-carboxamide (and isomers) are obtained.

The new compounds and processes according to the present invention allow for efficient syntheses of agrochemical and pharmaceutical compounds. The present processes for obtaining agrochemically or pharmaceutically active ingredients or intermediates thereof generally comprise fewer steps than currently available processes, allowing for economically and ecologically advantageous manufacture. Often, the process steps display good to excellent yields and selectivities. Recovery options of intermediates and auxiliaries, such as bases, are enhanced through such processes.

Should the disclosure of any patents, patent applications, and publications which are incorporated herein by reference conflict with the description of the present application to the extent that it may render a term unclear, the present description shall take precedence.

The following examples are intended to further explain the invention without limiting it.

### EXAMPLES

The starting materials are commercially available or known from literature procedures.

### Example 1a

### Manufacture of 4-(-2-benzylidene-1-methylhydrazinyl)but-3-en-2-one

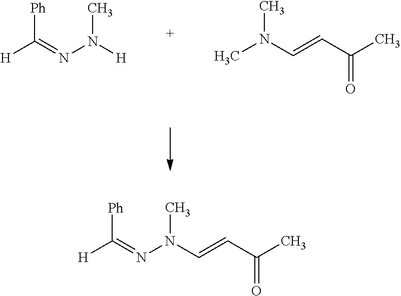

1 eq of 2-methyl-1-phenylhydrazone and 1.05 eq of 4-(dimethylamino)-but-3-en-2-one are charged in 20 mL of toluene, and heated to 40° C. The reaction mixture is stirred at 40° C. for about 8 hours. The solvent and dimethylamine is distilled off to yield the desired enone.

The presence of a Lewis acid, for example NaHSO₄ can enhance the reaction.

### Example 1b

### Manufacture of 4-(-2-benzylidene-1-methylhydrazinyl)but-3-en-2-one

1 eq of 2-methyl-1-phenylhydrazone and 1.05 eq of 4-ethoxybut-3-en-2-one are charged in 20 mL of toluene. The reaction mixture is stirred at 20° C. for about 4 hours. The volatiles are evaporated to yield the crude product.

### Example 2

### Manufacture of 3-((-2-benzylidene-1-methylhydrazinyl)methylene)-1,1-difluoropentane-2,4-dione

1 eq of the product of example 1b is reacted with 1.1 eq difluoroacetylfluoride in the presence of 1.2 eq triethylamine in 30 mL dichloromethane at 0° C. The reaction is continued for 2 hours at 25° C. The mixture is quenched with 50 mL of water, the aq. phase separated and extracted twice with dichloromethane. The organic phase is dried over Na2SO4, and the solvents are evaporated. Triethylamine can also be recovered from the aqueous phase after alkalising the aqueous phase and separation of the phases.

### Example 3

### Manufacture of 1-(3-(difluoromethyl)-1-methyl-1H-pyrazol-4-yl)ethanone

1 eq of 3-((-2-benzylidene-1-methylhydrazinyl)methylene)-1,1-difluoropentane-2,4-dione from example 2 is charged in 30 mL acetonitrile, and 0.5 eq of 10% HCl is added. The mixture is stirred at 20° C. for 1 hour. The solvent is removed, the mixture extracted with dichloromethane and dried over Na2SO4. Evaporation gives the crude product.

### Example 4

### Manufacture of (3-(difluoromethyl)-1-methyl-1H-pyrazol-4-carboxylic acid

1 eq of 1-(3-(difluoromethyl)-1-methyl-1H-pyrazol-4-yl)ethanone is added to 3.3 eq NaOH and 3.3 eq of an 8% sodium hypochlorite solution at 10° C. After addition, the reaction mixture is stirred for 3 hours at 20° C. Dichloromethane, about 20 mL, is added to the mixture and the organic phase discarded. The pH of the aq. phase is brought to 1-2 by addition of 10% HCl. The mixture is stirred for 30 minutes at 0° C., the precipitate filtered and dried to obtain (3-(difluoromethyl)-1-methyl-1H-pyrazol-4-carboxylic acid.

### Example 5

### Manufacture of 4-(2-benzylidene)hydrazinyl)but-3-en-2-one

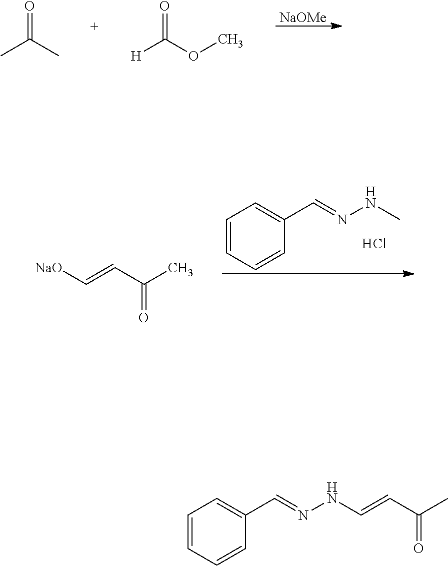

To a four necked flask 482 ml of toluene was charged and 48 ml of anhydrous methanol were added. 58.38 g (1.08 mol) of NaOMe was charged in one portion. A mixture of 72.72 g (1.2 mol) of methyl formate and 58.08 g (1 mol) of acetone was added dropwise in 2 hours, keeping reaction temperature at 35-40° C., then stirring was continued at 40° C. for 2 h. The mixture was cooled to 35° C. and 170.68 g (1 mol) of 1-benzylidene-2-methylhydrazine HCl salt (BzH*HCl) salt was added in one portion. The mixture was stirred at room temperature overnight. The salt was filtered of and the solution partly evaporated whereupon product 4-(2-benzylidene)hydrazinyl)but-3-en-2-one crystallized out. 4-(2-benzylidene)hydrazinyl)but-3-en-2-one was filtered off and dried yielding 183 g pale yellow crystals.

BzH*HCl was obtained by reaction of 1-benzylidene-2-methylhydrazine in ethyl acetate with HCl/dimethylether. During the addition, a white-yellow solid was formed. After 30 minutes at 0° C., the suspension was filtered and dried in vacuum to yield BzH*HCl. The synthesis of 1-benzylidene-2-methylhydrazine is described, for example, in A. Dubrovskiy et al J. Org. Chem., 2012, 77 (24), pp 11232-11256, and literature cited therein.

### Example 6

### Manufacture of 3-((2-benzylidene)hydrazinyl)methylene)-1,1-difluoropentane-2,4-dione

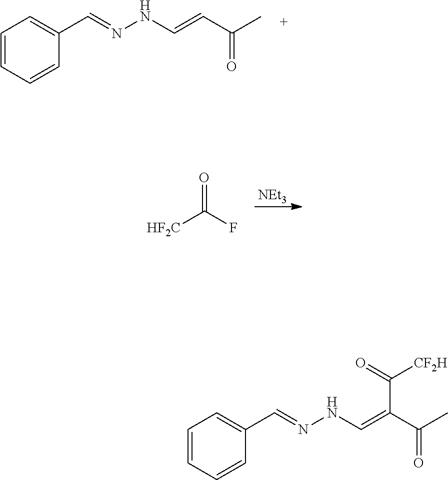

To a reaction flask was charged with 183 g 4-(2-benzylidene)hydrazinyl)but-3-en-2-one (0.9 mol) obtained in example 5. 1000 ml DCM was added following by 200 g Et₃N. Gaseous diflouroacetyl flouride (1 mol) was added to the mixture keeping internal temperature below 20° C. The mixture was stirred for 3 h whereupon GC showed full 4-(2-benzylidene)hydrazinyl)but-3-en-2-one consumption. The mixture was washed with water to remove the NEt₃ salt. The resulting solution was used directly in example 7.

### Example 7

### Manufacture of 1-(3-(difluoromethyl)-1-methyl-1H-pyrazol-4-yl)ethan-1-one

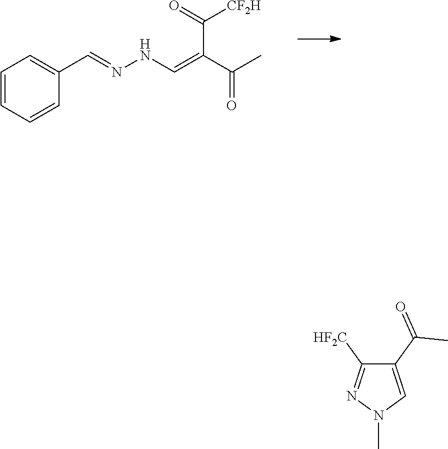

To the crude 3-((2-benzylidene)hydrazinyl)methylene)-1,1-difluoropentane-2,4-dione solution from example 6 was added conc. H₂SO₄ (25 g) and the mixture was stirred for 2 h at room temperature. The solution was washed with water to remove acid; the solvent was evaporated. Crude 1-(3-(difluoromethyl)-1-methyl-1H-pyrazol-4-yl)ethan-1-one containing benzaldehyde was separated by distilling benzaldehyde away under reduced pressure to yield crude 1-(3-(difluoromethyl)-1-methyl-1H-pyrazol-4-yl)ethan-1-one.

### Example 8

### Manufacture of (3-(difluoromethyl)-1-methyl-1H-pyrazol-4-carboxylic acid

1 eq of 1-(3-(difluoromethyl)-1-methyl-1H-pyrazol-4-yl)ethanone of crude 1-(3-(difluoromethyl)-1-methyl-1H-pyrazol-4-yl)ethan-1-one obtained by example 7 is added to 3.3 eq NaOH and 3.3 eq of an 8% sodium hypochlorite solution at 10° C. After addition, the reaction mixture is stirred for 3 hours at 20° C. Dichloromethane, about 20 mL, is added to the mixture and the organic phase discarded. The pH of the aq. phase is brought to 1-2 by addition of 10% HCl. The mixture is stirred for 30 minutes at 0° C., the precipitate filtered and dried to obtain (3-(difluoromethyl)-1-methyl-1H-pyrazol-4-carboxylic acid.

1. A process for the manufacture of a compound according to formula (I), 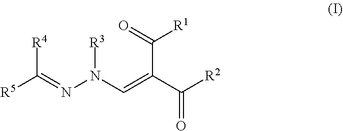
the process comprising a step of reacting a compound of formula (II) 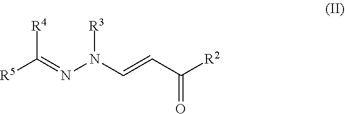
with a compound of formula (III) 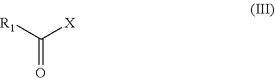
wherein
R¹ is selected from C₁-C₄-alkyl groups which may be substituted by one, two or three halogen atoms selected from the group consisting of F, Cl and Br, or by a CF₃ group
R² is selected from the group consisting of C₁-C₈-alkyl, aryl, C₃-C₈ cycloalkyl, aralkyl and heteroaryl, each of which is optionally substituted,
R³ is selected from the group consisting of C₁-C₁₂-alkyl, C₂-C₆ alkenyl, C₃-C₈ cycloalkyl, aryl, heteroaryl, aralkyl, each of which is optionally substituted; or R³ is a nitrogen protecting group
R⁴ and R⁵ independently from each other are selected from the group consisting of H, C₁-C₁₂-alkyl, C₃-C₈ cycloalkyl which optionally contains one or two heteroatoms selected from the group consisting of N, O and S, aryl and heteroaryl, each of which is optionally substituted; or R⁴ and R⁵ together with the carbon atom to which they are attached form a 4, 5 or 6-membered optionally substituted cycloalkyl, which optionally contains one or two heteroatoms selected from the group consisting of N, O and S, aryl or heteroaryl group, each of which is optionally substituted,
X is a halogen atom;
or with a respective anhydride (Ma) of the compound of formula (III).

2. A process for the manufacture of a compound according to formula (II), 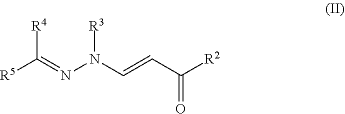
the process comprising a step of reacting a compound of formula (IV) and a compound of formula (V) 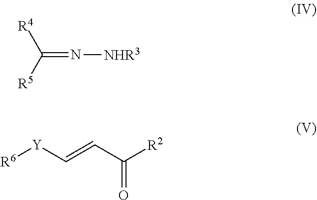
wherein
R¹ is selected from C₁-C₄-alkyl groups which may be substituted by one, two or three halogen atoms selected from the group consisting of F, Cl and Br, or by a CF₃ group
R² is selected from the group consisting of C₁-C₈-alkyl, aryl, C₃-C₈ cycloalkyl, aralkyl and heteroaryl, each of which is optionally substituted
R³ is selected from the group consisting of C₁-C₁₂-alkyl, C₂-C₆ alkenyl, C₃-C₈ cycloalkyl, aryl, heteroaryl, aralkyl, each of which is optionally substituted; or R³ is a nitrogen protecting group
R⁴ and R⁵ independently from each other are selected from the group consisting of H, C₁-C₁₂-alkyl, C₃-C₈ cycloalkyl which optionally contains one or two heteroatoms selected from the group consisting of N, O and S, aryl and heteroaryl, each of which is optionally substituted; or R⁴ and R⁵ together with the carbon atom to which they are attached form a 4, 5 or 6-membered optionally substituted cycloalkyl, which optionally contains one or two heteroatoms selected from the group consisting of N, O and S, aryl or heteroaryl group, each of which is optionally substituted,
Y is selected from the group consisting of S, O and NR⁷,
R⁷ and R⁶ independently are selected from the group consisting of C₁-C₁₂-alkyl, C₂-C₆ alkenyl or C₃-C₁₀-cycloalkyl, each of which is optionally substituted,
or, when Y═NR⁶, R⁶ together with R⁷ and the nitrogen atom to which the two radicals are attached are an optionally substituted 5- to 10-membered heterocyclic radical which, in addition to the nitrogen atom, may contain a further 1, 2 or 3 heteroatoms selected from the group consisting of O, N and S as ring members.

3. The process of claim 1, wherein R¹ is methyl or ethyl, which is substituted by at least one fluorine atom.

4. The process according to claim 1, wherein R¹ is selected from the group consisting of CF₂Cl, CF₂H, CFCl₂, CFClH, CF₂Br, CF₂CF₃ and CF₃.

5. The process according to claim 1, wherein R² is a C₁-C₈-alkyl group, which is optionally substituted.

6. The process according to claim 1, wherein R⁴ is a hydrogen atom and R⁵ is selected from the group consisting of C₁-C₁₂-alkyl, C₃-C₈ cycloalkyl, aryl and heteroaryl, each of which is optionally substituted.

7. The process according to claim 1, wherein R³ is a methyl group.

8. A process for the manufacture of a compound of formula (VI) 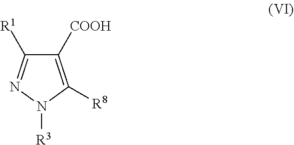
wherein R¹ and R³ have the same meaning as above, and R⁸ is selected from the group consisting of H, X′, COOR′, OR′, SR′, C(O)NR′₂, wherein R′ is hydrogen or a C₁-C₁₂-alkyl group, CN, C₁-C₁₂-alkyl, C₂-C₆ alkenyl, aryl, cycloalkyl, aralkyl, heteroaryl, each of which is optionally substituted, with the proviso that in C(O)NR′₂ both R′ may be the same or different;
the process comprising a step of performing the process according to claim 1.

9. The process according to claim 8, wherein the process comprises additionally at least one of the steps of
(a) adding an acidic compound to the compound of formula (I) to obtain a compound of formula (VII) 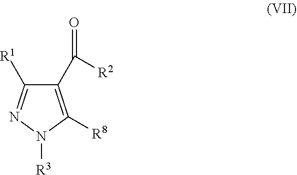
(b) reacting a compound of formula (VII) with at least one oxidizing agent, wherein R¹, R³, R⁸ and R² have the same meaning as given above.

10. The process according to claim 9, wherein the at least one oxidizing agent is selected from the group consisting of halogen, oxyacids of halogen and salts thereof, a peroxide, molecular oxygen and molecular ozone.

11. The compound of formula (II) as defined in the process of claim 1.

12. Compound A compound of formula (I), 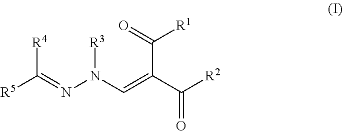
wherein
R¹ is selected from C₁-C₄-alkyl groups which may be substituted by one, two or three halogen atoms selected from the group consisting of F, Cl and Br, or by a CF₃ group
R² is selected from the group consisting of C₁-C₈-alkyl, aryl, C₃-C₈ cycloalkyl, aralkyl and heteroaryl, each of which is optionally substituted,
R³ is selected from the group consisting of C₁-C₁₂-alkyl, C₂-C₆ alkenyl, C₃-C₈ cycloalkyl, aryl, heteroaryl, aralkyl, each of which is optionally substituted; or R³ is a nitrogen protecting group
R⁴ and R⁵ independently from each other are selected from the group consisting of H, C₁-C₁₂-alkyl, C₃-C₈ cycloalkyl which optionally contains one or two heteroatoms selected from the group consisting of N, O and S, aryl and heteroaryl, each of which is optionally substituted; or R⁴ and R⁵ together with the carbon atom to which they are attached form a 4, 5 or 6-membered optionally substituted cycloalkyl, which optionally contains one or two heteroatoms selected from the group consisting of N, O and S, aryl or heteroaryl group, each of which is optionally substituted.

13. (canceled)

14. A process for the manufacture of an agrochemical or pharmaceutical compound or their intermediates, the process comprising a step of performing the process according to claim 1.

15. The process according to claim 1, wherein the compound of formula (II) is obtained by the reaction of a compound of formula (IX) with a compound of formula (IV) 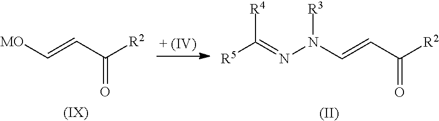
wherein R² to R⁵ and (IV) are defined as in claim 1, and wherein M is a metal ion.

16. The process of claim 3, wherein R¹ is methyl, which is substituted by at least one fluorine atom.

17. The process according to claim 5, wherein R² is a methyl group which is optionally substituted.

18. The process according to claim 9, wherein the acidic compound is selected from the group consisting of HCl, H₂SO₄, KHSO₄ and HF.

19. The process according to claim 9, wherein the compound of formula (VII) is reacted with at least one oxidizing agent in the presence of a base.

20. The process according to claim 10, wherein the at least one oxidizing agent is selected from the group consisting of halogen and aqueous solutions of salts of hypohalogenous acids.

21. The process according to claim 20, wherein the at least one oxidizing agent is an aqueous solution of sodium hypochlorite and/or sodium hypobromite.
